(19) United States
(12) Patent Application Publication
(10) Pub. No.: US 2007/0104801 A1
(43) Pub. Date: May 10, 2007

(54) Fertility and anti-aging supplement for the fertility health of females and males

(75) Inventors: Michael D. Cecchi, Madison, CT (US); Monica Mezezi, Guelph (CA); Klaus Weimer, Kirkland, WA (US)

Correspondence Address: William W. Jones, 6 Juniper Lane, Madison, CT, 06443, US

(73) Assignee: IVF Online LLC

(21) Appl. No.: 11/267,267

(22) Filed: Nov. 7, 2005

(51) Int. Cl. A61K 36/185; A61K 31/525; A61K 33/26; A61K 33/04; A61K 33/32; A61K 36/82; A61K 36/87

(52) U.S. Cl. 424/641; 424/725; 424/729; 424/766; 514/54; 514/62; 424/702; 424/646; 514/251; 514/52; 514/350

(57) ABSTRACT

A fertility-enhancing supplement contains a balance of compositions and ingredients that will enhance the reproduction system in the female and male to help support conception, while also supporting the anti-aging of the reproductive system, taken in balance to aid in the uptake by the body of certain and particular compositions that are included in this invention. The supplement can include ingredients to assist the female reproductive system to conceive and in the male to aid in the production of more healthy sperm. Such ingredients as glucosamine sulfate are included separately or in conjunction to help the female reproductive system to maintain a youthfulness in the female and are important to the maintaining of the vitality of eggs and reducing the aging process in the development of fertilized eggs, embryos and facilitating the implantation of embryos. The supplement will help in the preservation of the eggs and the reproductive system and the overall reproductive health, as the female ages, and assist the male in sperm production throughout the child bearing years.

### TECHNICAL FIELD

The invention is a composition of nutritional supplements that will enhance the fertility health of both males and females humans, while maintaining an anti-aging regiment of ingredients that will maintain the reproductive system as the person ages. By providing these supplements, the invention will add specific enhancers that may be deficient in the reproductive system of both men and woman. The invention will provide, unlike others, a continued regimen of supplements that will provide the reproductive system with a continued supply of herbs, minerals, vitamins and antioxidants that will enter the reproductive system and will provide ingredients and building block substances, where in the body will manufacture certain agents that will boost the reproductive system. The invention will provide certain ingredients that will supply the reproductive system with certain ingredients which will assist in the reducing or slowing down the aging process of the reproductive systems as well. The invention will also provide to the system ingredients which will immediately aid the reproductive system and will assist the user to become pregnant at this time. The invention will be predicated on a continued regimen that will enhance the current possibilities of pregnancies, but will also aid in the preservation of the reproductive system for the future of women as their system and eggs begin to age. The invention will also relate to the ingredient levels of all components.

### BACKGROUND ART

Some 10 million individuals and families suffer from infertility or problems related to the inability to conceive or have offspring. These conditions may be present in either male or female individual.

Infertility problems may be caused by genetic or physical altercations and may also be created by eating habits and consumption or lack of consumption of certain vitamins, minerals and herbs. The consumption of nicotine from smoking, caffeine, drug, alcohol consumption, environmental toxins, and stress are all related to the infertility problems in men and women.

Another problem which causes infertility is age. As the female ages, so do her reproductive system, reproductive tract, uterus and the eggs within this system. As the male ages his production of sperm and motile sperm is reduced and this reduces his ability to contribute naturally to the conception process.

Nutrients within the reproductive system may also begin to diminish and be less prevalent as time goes on. These nutrients, such as Vitamin E and Folic Acid have been linked to reduced conception rates.

The lack or reduction of these nutrients will also impact the overall possibility of conception. Such nutritional supplements as vitamins B6, B12, and E, folic acid, calcium, along with herbs such as Vitax (chasteberry) and selenium have been linked to better fertility health.

Some of these supplements may also be related to the overall health of the embryos and the reduction of birth defects if in fact they are supplemented to the female, during the period in which she if trying to conceive. Most of the supplemental regimens, do however only address the need for these supplements during the period in which the female is trying to conceive, and do not address the overall preservation of the female reproductive system for the future.

Women planning child bearing later in life must also consider the supplementation of the reproductive system, uterus, reproductive tract and eggs within this system during the time in which they are not trying to immediately conceive.

If a woman defers child bearing into her later years, due to career options or other reason, she does not consciously maintain a regimen of supplements that will maintain the reproductive system, and the “youth” of such and will be less likely to conceive when the time comes.

Woman in the ages of 20 to 27 are most likely at the best time to naturally conceive, aside form any obvious fertility problems. As they age to 30 and then to 40, for those who may have only deferred their child bearing years for other reasons than infertility, the likelihood of becoming pregnant greatly diminishes with each year. During this time the reproductive system may also be reduced in its ability to conceive due to the introduction of nicotine, alcohol or environmental pollutants and such through the consumption habits of the individual and the environment they are in.

Our recent study shows the following decline in a woman's fertility as she ages and an increase in her possibilities of infertility.
Age group: 17-20
Possibility of becoming pregnant (%): 90
Possibility of being infertile (%): 3
Age group: 21-25
Possibility of becoming pregnant (%): 85
Possibility of being infertile (%): 5
Age group: 26-30
Possibility of becoming pregnant (%): 80
Possibility of being infertile (%): 10
Age group: 31-35
Possibility of becoming pregnant (%): 65
Possibility of being infertile (%): 15
Age group: 36-40
Possibility of becoming pregnant (%): 50
Possibility of being infertile (%): 20
Age group: 41-45
Possibility of becoming pregnant (%): 35
Possibility of being infertile (%): 35
Age group: 46-50
Possibility of becoming pregnant (%): 5
Possibility of being infertile (%): 65
Age group: 51 and over
Possibility of becoming pregnant (%): 0
Possibility of being infertile (%): 100

The above percentages are rounded for this presentation and show a statistical average bases on a grouping of various source information. The two factored results, “Possibility of becoming pregnant”, and the “Possibility of being infertile” are independent of each other and due to additional factors may not add up to 100%.

As the woman ages, it is scientifically proven that the reproductive tract, uterus and eggs also ages. The women moves into menopause and this contributes to the system change and degeneration. As a result of the aging process the zona pollucida or shell of the egg become harder and do not allow the sperm to easily penetrate upon contact. This hardening also impedes the eggs from expanding upon fertilization and also result in lack of conception. Older women and older eggs may also result in other abnormalities or defects after child birth.

Present dietary supplements do not take into consideration conditions which may be directly related to the needs of the reproductive tract, uterus or eggs during this time when child bearing has been deferred.

Studies indicate that hyaluronic acid and glucosamine may be present in the outer shell of the oocytes or preservation of quality of eggs in the reproductive tract.

The prior art does not address the deficiencies of certain supplements which may be taken over a short period or over a greater period of time to aid the eggs within the reproductive tract or to support the reproductive tract and uterus over time.

The prior art does not address the reduction of the quality of the eggs as the female reproductive tract begins to age, as the female ages, and supporting the nutritional needs of this system and aiding in maintaining a healthier level of ingredients which will keep the reproduction system to remain in a more youthful mode and not to deteriorate as it would if not supplemented.

It would be highly desirable to use a combination of nutritional supplements which help to support the reproductive tract and uterus as a female ages and to help maintain the quality of the eggs within the reproductive tract as they become less viable and useful over the years. It would also be highly desirable to use a combination of nutritional supplement which helps to introduce certain supplements into the reproductive tract and uterus thereby maintaining the quality of the eggs and the uterus ooccte within the reproductive tract to help it remain for more viable and useful over the years.

It would be highly desirable for a combination of supplement which helps to introduce certain other supplements into the reproductive tract and uterus thereby maintaining or increasing the quality of the eggs within the reproductive tract to become more viable and useful over the years, and to be more viable after they have been extracted or fertilized.

It would be highly desirable for a combination of nutritional supplement which helps to support the reproductive tract and uterus over a longer period of time and to help to introduce as an ingredient glucosamine in various amounts.

It would be highly desirable for a combination of nutritional supplement which helps to support the reproductive tract and uterus over a longer period of time and to help to introduce as an ingredient hyaluronic acid in various amounts.

### DESCRIPTION OF THE INVENTION

This invention relates to dietary supplements of vitamins, mineral, herbs and antioxidants which will help to enhance the reproductive system of a female and to maintain a youthfulness to this system and eggs, as the female ages.

The invention relates to the composition of supplements that have been shown to aid in the conception process and to contain critical nutritional supplements in safe and effective doses that will optimize the possibility of conception and childbearing.

The composition is intended to be administered over the years of the woman's life from the approximate ages of 20 to 45. The composition is intended to add key nutrients for the immediate conception possibilities, but more importantly to help maintain the youthfulness of the reproductive system, uterus and eggs within the system for the reproductive life of the individual over a longer period of time.

The invention takes into consideration the intake, uptake and natural building process of the body to help to support the reproductive system for the life of the female. The combination consists of vitamins, minerals, herbs and additional ingredients which when take in combination work together to result in a greater uptake of certain ingredients and to support certain additional ingredients that become the building blocks for the body to synthesize certain other nutrients for use.

One such example of this is the inclusion of glucosamine. The glucosamine may be synthesized into hyaluronic acid and then taken to the key areas of the reproductive tract and uterus, from the intake and then from the uptake into the system. While the glucosamine, on the intake, becomes the building block for the body to naturally synthesize hyaluronic acid for the longer uptake and more natural composition for better absorption, resulting in a longer period of time for the body to synthesize and utilize the hyaluronic acid.

The composition of ingredients includes vitamins, minerals and antioxidants which work together to accomplish the goals of the overall supplements.

Key ingredients for the anti-aging process are alpha-lipoic add, beta-carotene, Glucosamine Sulfate and as key ingredients for the aiding of the fertility process are folic acid, iron, Inositol selenium, zinc, chasteberry (Vitex), Coenzyme Q10, as antioxidants ingredients are green tea, grape seed extract along with an aphrodisiac such as peruvian maca.

Certain other ingredients may be omitted, that are present in the prior art or in conventional theory, in that they are already present in many food sources and may also be present in the daily vitamin supplements the individuals are taking and therefore may not be necessary and we have included as not to create an overdose or excessive dose problem for the individual.

One embodiment of the invention will include Vitamin B6, B12, E, folic add, iron, calcium, selenium, zinc, L-arginine, chasteberry (Vitex), antioxidants in the form of green tea, ginseng, hyaluronic acid and glucosamine sulfate.

Another embodiment of the invention will include vitamins B6, B12, E, folic add, iron, calcium, selenium, zinc, L-arginine, chasteberry (Vitex), antioxidants in the form of green tea, Siberian ginseng, hyaluronic acid and not glucosamine sulfate.

Another embodiment of the invention will include vitamins B6, B12, E, folic acid, iron, calcium, selenium, zinc, L-arginine, chasteberry (Vitex), antioxidants in the form of green tea, Siberian ginseng, glucosamine sulfate and not hyaluronic add.

The various embodiments are a result of difference physiological needs of individuals and changes in ingredient levels due to these different individual needs. The ingredients for the composition have shown to aid in conception and the individual ingredients have shown to promote the following:

Vitamin B6 and B12 have been shown to be a nutrient in female reproductive systems for hormone metabolism, sperm formation and motility. Vitamin E and selenium are all potent antioxidants that help improve the body.

Folic acid is the precursor for 5-methyltetrahydrofolate, a coenzyme that functions as a methyl donor in many synthetic reactions. Folate deficiency during pregnancy has been linked to fetal neural tube defects and therefore is recommended in all pregnant women, and women attempting to become pregnant.

Iron, calcium and zinc are added to help supplement any deficiencies and are more common ingredients.

Vitex agnus-castus, also known as chasteberry has been know to improve hormone balance by increasing progesterone release and ovulation frequency. Green tea is used as an antioxidant.

Glucosamine sulfate is the building block for hyaluronic acid and will help to maintain longevity of the production of hyaluronic acid in the body and the reproductive system.

Hyaluronic acid is a polysaccharide that is found in joint tissue and in the vitreous humor of the eye. Hyaluronic acid functions as a protective coating and a lubricant for soft tissue and joints, and additionally, helps maintain the structural integrity of soft tissue. R has also been scientifically proven to be in the reproductive tract and uterus, along with its help in the implantation of embryos.

Grape seed extract (derived from grape seeds) contains a number of unique beneficial flavonoid compounds which help to protect cells from free radical damage and promote healthy circulation. Easily digested and absorbed, and remains effective for up to 20 times longer than other antioxidants, working harder and longer

Alpha-lipoic acid, (ALA) is a potent and “universal” antioxidant, that can travel to each and every cell of the body, to fight Free Radical Oxidation. Alpha lipoic acid protect the mitochondria and the genetic material, DNA. As we age, it naturally enhances the way our body uses B and E vitamins, and other antioxidants, “recycling” E and C and thus making them much more effective. ALA is also vital to the production of energy for the body, and stimulates production of glutathione.

Beta-Carotene is a source of vitamin A, and powerful antioxidant Beta-carotene strengthens the immune system by neutralizing free radicals that damage cells. By directly combating cell damage it may even reverse some disorders. It has been shown to exhibit these antioxidant properties in the skin, colon, prostate, uterus and cervix, among other areas.

Peruvian Maca has shown some benefit to woman and men of all ages, promoting production of new hormones depleted by age, increase energy level, treats PMS, increases libido; improves erectile function and general well being.

Horny Goat Weed is an herb, with testosterone-like quality, for sexuality and energy, aphrodisiac, stimulates sexual activity in men, increases sperm production, sexual desire, and performance.

Overall, the antioxidants, green tea, vitamin E, and selenium, improve overall reproductive health. L-arginine, stimulates the reproductive organs by improving circulation. Folic acid, vitamins B6 and B12, iron, zinc and magnesium help promote women's' fertility.

The invention will become more apparent from a review of the tables below, which show the preferred and desired ranges of the compositon's ingredients.

TABLE 1
Composition 1 for fertility and anti-aging for women:

| | | |
|---|---|---|
| Component | Preferred Dose | Dose Range |

| Alpha-lipoic acid | 50 | mg/day | 10-100 | mg/day |
|---|---|---|---|---|
| Beta-Carotene | 500 | IU | 100-1000 | IU |
| Vitamin B6 | 6 | mg/day | 2-12 | mg/day |
| Vitamin B12 | .0024 | mg/day | .0010-.0048 | mg/day |
| Vitamin E | 200 | IU | 100 IU-800 IU | |
| Folic Acid | 0.4 | mg/day | .2-1.0 | mg/day |
| Iron | 10 | mg/day | 2-20 | mg/day |
| Calcium | 100 | mg/day | 50-400 | mg/day |
| Manganese | 2 | mg/day | 1-5 | mg/day |
| Selenium | 0.06 | mg/day | .02-.08 | mg/day |
| Zinc | 8 | mg/day | 2-40 | mg/day |
| chasteberry | 40 | mg/day | 10-80 | mg/day |
| (Vitex 10:1 extract) | | | | |
| Inositol | 10 | mg/day | 2-20 | mg/day |
| Green Tea | 10 | mg/day | 2-50 | mg/day |
| Peruvian Maca | 200 | mg/day | 50-400 | mg/day |
| Glucosamine Sulfate | 200 | mg/day | 50-800 | mg/day |
| Grape Seed extract | 20 | mg/day | 5-50 | mg/day |

Composition 2 for fertility and anti-aging for women:

| | | |
|---|---|---|
| Component | Preferred Dose | Dose Range |

| Vitamin B6 | 6 | mg/day | 2-12 | mg/day |
|---|---|---|---|---|
| Vitamin B12 | .0024 | mg/day | .0010-.0048 | mg/day |
| Vitamin E | 200 | IU | 100 IU-800 IU | |
| Folic Acid | 0.4 | mg/day | .2-1.0 | mg/day |
| Iron | 10 | mg/day | 2-20 | mg/day |
| Calcium | 200 | mg/day | 50-400 | mg/day |
| Selenium | 0.06 | mg/day | .02-.08 | mg/day |
| Zinc | 8 | mg/day | 2-40 | mg/day |
| chasteberry | 40 | mg/day | 10-80 | mg/day |
| (Vitex 10:1 extract) | | | | |
| Green Tea | 20 | mg/day | 2-50 | mg/day |
| Glucosamine Sulfate | 400 | mg/day | 50-800 | mg/day |

TABLE 2
Composition 1 for fertility and anti-aging for men:

| | | |
|---|---|---|
| Component | Preferred Dose | Dose Range |

| Alpha-lipoic acid | 50 | mg/day | 10-100 | mg/day |
|---|---|---|---|---|
| Beta-Carotene | 500 | IU | 100-1000 | IU |
| Vitamin B12 | .002 | mg/day | .0010-.0048 | mg/day |
| Vitamin C | 250 | IU | 100 IU-2000 IU | |
| Vitamin E | 250 | IU | 100 IU-2000 IU | |
| Folic Acid | 0.4 | mg/day | .2-1.0 | mg/day |
| Acetyl-L-Carnitine | 750 | mg/day | 200-1500 | mg/day |
| L-Arginine | 2000 | mg/day | 500-4000 | mg/day |
| Selenium | 0.15 | mg/day | .10-.30 | mg/day |
| Zinc | 10 | mg/day | 2-40 | mg/day |
| chasteberry | 40 | mg/day | 10-80 | mg/day |
| (Vitex 10:1 extract) | | | | |
| Inositol | 10 | mg/day | 2-20 | mg/day |
| Green Tea | 15 | mg/day | 2-40 | mg/day |
| Peruvian Maca | 200 | mg/day | 50-400 | mg/day |
| Glucosamine Sulfate | 200 | mg/day | 50-400 | mg/day |
| Grape Seed extract | 10 | mg/day | 5-50 | mg/day |

Composition 2 for fertility and anti-aging for men:

| | | |
|---|---|---|
| Component | Preferred Dose | Dose Range |

| Vitamin B12 | .002 | mg/day | .001-.005 | mg/day |
|---|---|---|---|---|
| Vitamin C | 250 | IU | 100 IU-400 IU | |
| Vitamin E | 250 | IU | 100 IU-400 IU | |
| Folic Acid | 0.4 | mg/day | .1-1.0 | mg/day |
| Acetyl-L-Carnitine | 750 | mg/day | 200-1500 | mg/day |
| L-Arginine | 2000 | mg/day | 500-4000 | mg/day |
| Selenium | 0.15 | mg/day | .10-.30 | mg/day |
| Inositol | 10 | mg/day | 2-20 | mg/day |
| Green Tea | 15 | mg/day | 2-50 | mg/day |
| Peruvian Maca | 200 | mg/day | 50-400 | mg/day |
| Horny Goat | 200 | mg/day | 50-400 | mg/day |

While the invention has been described with references to specific embodiments, modifications and variations of the invention may be constructed without departing from the scope of the invention, which is defined in the following claims.

What is claimed is:

1. A fertility-enhancing composition of ingredients for promoting fertility health in a women and men, as well as promoting anti-aging processes of the reproductive system.

2. The fertility-enhancing composition in claim 1 including an effective amount of alpha-lipoic-acid.

3. The fertility-enhancing composition in claim 1 including an effective amount of glucosamine sulfate.

4. The fertility-enhancing composition in claim 1 including an effective amount of Peruvian maca.

5. The fertility-enhancing composition in claim 1 including an effective amount of Peruvian maca and homey goat weed.

6. A fertility-enhancing composition of ingredients that promotes fertility in the female, said composition comprising Vitex agnus-castus (chasteberry), selenium, folic acid, vitamin B6, vitamin B12, iron, zinc, and magnesium, ferulic add, green tea, vitamin E, hyaluronic add and glucosamine sulfate.

7. A fertility-enhancing and anti-aging supplement which contains the composition hyaluronic acid.

8. A fertility-enhancing supplement which contains the composition hyaluronic acid, where the hyaluronic acid is derived from glucosamine sulfate.

9. A fertility-enhancing supplement which contains glucosamine sulfate.

10. A fertility-enhancing supplement, comprising Vitex agnus-castus (chasteberry), antioxidants, green tea, vitamin E, and selenium, L-arginine, an amino acid for stimulating reproductive organs by improving circulation, folic acid, vitamins B6 and B12, iron, zinc, and glucosamine for promoting a women's fertility and maintaining healthier eggs, uterus and reproductive system.

11. The fertility-enhancing supplement of claim 10 further comprising hyaluronic acid.

12. A fertility-enhancing compositon of ingredients for promoting fertility health in a women and men, as well as promoting anti-aging processes of the reproductive system, comprising alpha-lipoic acid, grape seed acid and Vitex agnus-castus (chasteberry).

13. A fertility-enhancing composition of ingredients for promoting fertility health in a women and men, as well as promoting anti-aging processes of the reproductive system, comprising alpha-lipoic acid and Vitex agnus-castus (chasteberry).
